(19) United States
(12) Patent Application Publication
(10) Pub. No.: US 2023/0172337 A1
(43) Pub. Date: Jun. 8, 2023

(54) MAGNETIC ARTIFICIAL FINGERNAIL EXTENSIONS

(71) Applicant: Anna Rose Richwine, Santa Clarita, CA (US)

(72) Inventor: Anna Rose Richwine, Santa Clarita, CA (US)

(21) Appl. No.: 17/543,486

(22) Filed: Dec. 6, 2021

(51) Int. Cl. A45D 31/00; A61K 8/19; A61Q 3/02

(52) U.S. Cl. CPC A45D 31/00; A61K 8/19; A61Q 3/02; A45D 2200/20

(57) ABSTRACT

Disclosed herein is an artificial fingernail that magnetically affixes to the fingers of a user. The user first applies a clear magnetic nail polish to their fingers. When dry, the artificial fingernail, manufactured including magnetic polymers, affixes directly to the magnetic nail polish coating. In various embodiments, the magnetic polymers used in the artificial fingernail are configured to various contexts and preferences.

### TECHNICAL FIELD

The disclosure relates to disposable and reusable artificial cosmetic fingernails.

### BACKGROUND

Nail extensions or artificial nails are a leading industry when it comes to beauty consumer products. Artificial nails, also known as fake nails, false nails, fashion nails, acrylic nails, nail extensions or nail enhancements, are extensions placed over fingernails as fashion accessories. Some artificial nail designs attempt to mimic the appearance of real fingernails as closely as possible, while others may deliberately stray in favor of an artistic look. Artificial nails tend to appeal to “do-it-yourselfers” and tend to be easy to apply, though unlike most manicures, artificial nails require regular upkeep. Nonetheless, their versatility in terms of shape, size, design and comparatively high durability are some advantages they hold over other types of manicures. Notably, known artificial nails require the use of glues that may be damaging to existing fingernails.

### BRIEF DESCRIPTION OF THE DRAWINGS

FIG. 1 is a perspective view illustrating a prior art procedure wherein the adhesive is applied rearward of the nail extension.

FIG. 2 is a flowchart illustrating the use of magnetic artificial fingernails.

FIGS. 3A-D are depictions of a number of varied configurations of magnetic polymer artificial nails wherein:

FIG. 3A depicts a sharp change in magnetic moment of the artificial magnetic nail;

FIG. 3B depicts a gradual change in magnetic moment of the artificial magnetic nail;

FIG. 3C depicts a couple anchor points of magnetic field of the artificial magnetic nail, and

FIG. 3D depicts a number of anchor points of magnetic field distributed throughout the artificial magnetic nail.

### DETAILED DESCRIPTION

Disclosed herein are artificial fingernail extensions made from a magnetic polymer and paired with a magnetic nail polish. The artificial nails are attached to a wearer through magnetism and may be freely removed, exchanged with other artificial nails, without damaging the underlying real fingernail.

Recent development and characterization of stable polymer formulations for manufacturing magnetic composites are currently being implemented. Permanent bonded magnets are used in some consumer and industrial applications that involve the conversion of mechanical energy to electrical energy, and vice-versa. Magnetic Polymer finds applications in areas like factory automation, medical devices, household appliances, consumer electronics, and automotive systems. Among many permanent magnetic materials, alnico, ferrites, samarium cobalt, and neodymium iron boron (Nd₂Fe₁₄B, abbreviated herein as NdFeB) are predominantly used in the industry. The binders include polyamide (PA), polytetrafluoroethylene, epoxies, polyester and polyphenylene sulfide. Four traditional processes utilized to manufacture bonded magnets are extrusion, compression molding, injection molding and calendaring.

The manufacturing process uses magnetic nanoparticles, such as neodymium earth magnets, cobalt, and nickel are injection molded into the artificial nail form. The artificial nail is a hardened magnetic polymer. During manufacturing a degree of magnetism is determined based on the amount/concentration of magnetic particles used. In some embodiments, the artificial nail mold is constructed to have multiple sections where different concentrations of magnetic particles are used.

For example, in some embodiments, magnetic particles are be concentrated at the base of the artificial nail and be present at a lower concentration, or not at all, at the tip of the artificial fingernail. During manufacture, the magnetic polymer is dried in a presence of a hard permanent magnet in order to align the nanoparticles polarity in a particular direction (e.g., to prevent nanoparticles from pairing up with one another and reducing the attractive strength of the magnetic field of the overall structure).

In some embodiments, the clear nail polish is manufactured in a similar process but left as a liquid that air dries. In embodiments that make use of hard magnetic particles, such as NIB magnets, application of the nail polish includes drying in the presence of other hard magnets, such as the artificial nail itself. However, some embodiments make use of soft magnets (e.g., magnets that have a low coercive force, such as nickel/iron alloys). An example of a magnet with a low coercive force and a high moment is permalloy. In embodiments where the nail polish includes nanoparticles of a soft magnet, the artificial nail (a hard magnet) is sufficient align the magnetic field of the nail polish and attract thereto.

Existing magnetic nail polishes are used to develop distinctive ornamental designs. Specifically, colored nail polish including magnetic particles is painted on a nail, and prior to drying, a magnet is waved over the nail. The magnet draws the particles through the wet nail polish to the top surface and alters the configuration of the ornamental appearance. The nail polish preparation disclosed herein does not inherently include inclusion of an ornamental purpose, though one is able to incorporate such ornamental features as desired.

Embodiments of the magnetic nail polish that make use of soft magnets disclosed herein operate differently. Instead, the nail polish is applied and allowed to dry. The polarity of the magnetic particles in the liquid state of the nail polish is irrelevant because once dry, the presence of a hard magnet is sufficient to coerce the magnetic field as necessary. In embodiments that use hard magnetic nanoparticles in the nail polish, a permanent magnet is held over the applied, wet nail polish to align the magnetic particles. Alignment does not require that the particles be pulled to the surface. Alignment of the magnetic field serves to better affix the artificial nail.

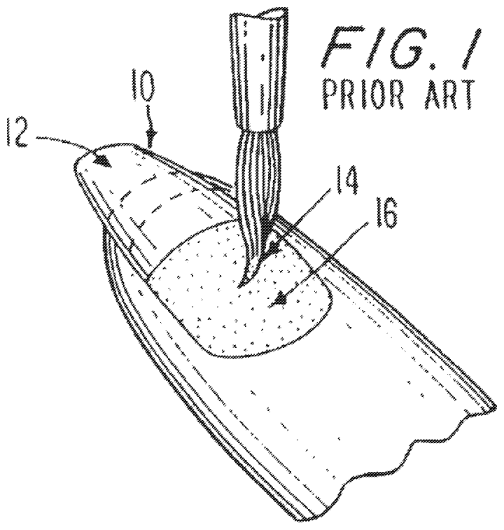

FIG. 1 is a perspective view illustrating a prior art procedure wherein an adhesive is applied rearward of the nail extension. FIG. 1 depicts the prior art artificial fingernail 10 described in U.S. Pat. No. 4,450,848 to Ferrigno. In FIG. 1, a plastic extension or tip 12 is adhered to the end of the natural fingernail using an adhesive. The figure depicts the method employed in Ferrigno whereby liquid cyanoacrylate adhesive 14 is then applied to the top surface 16 of the natural fingernail only rearward of the tip 12. Following this, a standard acrylic ester copolymer in powder form is also applied and allowed to dry. In Ferrigno, the form used on the tip (extension) is made of acetate plastic. Other methods of the prior art include the application of adhesive to the top surface of a natural fingernail and to the top surface of a fingernail extension.

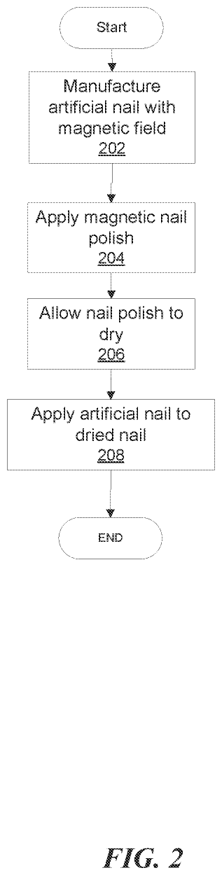

FIG. 2 is a flowchart illustrating the use of magnetic artificial fingernails. In step 202, the artificial nail(s) are manufactured such as though injection molding. Injection molding uses existing molds that receive molten polymer and set as a solid. The polymers used include small magnetic particles. During manufacture, the molten polymer is allowed to dry in the presence of a hard magnet that aligns the magnetic particles of the polymer in one direction.

Left on their own, magnetic polymers within a liquid solution will pair up, aligning in opposite directions such that the resulting material negates the magnetic field in an antiferromagnetic configuration (e.g., zero net magnetic moment). When dried in the presence of a hard magnet, the magnetic polymer has a ferromagnetic configuration (e.g., a combined magnetic moment).

During manufacture, the strength, or moment of the magnetic polymer is configured. There are multiple ways of varying the strength of the magnetic field. In some embodiment, the materials used for the magnetic particles are chosen for their moment. NIB magnets are well-known as having a high magnetic moment. Other materials have varying moments and may be selected as desired. In some embodiments, the strength of the magnet is configured by the concentration of the magnetic particles within the polymer. An increase in magnetic material increases the overall moment of the resulting polymer.

In some embodiments the artificial nail is manufactured with different moments at different locations of the artificial nail. For example, at the base of the artificial nail, which corresponds with the base of the fingernail upon which the artificial nail is applied, the magnetic moment is configured to be higher than the tip of the artificial nail. By varying the moment of the magnetic field the attractive force of the tip of the nail is lower than the base. Lower attractive force at the tip of the nail reduces discomfort of the wearer as their hand approaches external metallic materials (e.g., a car door, a refrigerator).

During the injection molding process, the mold itself may be divided into subsections, and/or known volumes of molten magnetic polymer that have different moments (by any of concentration and/or material choice) are injected in a predetermined order into the mold.

The number of varied magnetic polymer solutions used during manufacturing modifies the manner of change of the magnetic field. If two types are used, there will be a sharp contrast therebetween. If many types are used there may be a gradient of magnetic moments of the artificial nail.

In some embodiments, the nail is configured to have multiple sections of magnetic polymer interspersed with non-magnetic material. For example, the artificial nail includes magnetic polymer at the base of the nail, followed by a section that is non-magnetic, then another magnetic section that corresponds to a predicted location of the fingertip (e.g., the end of the existing fingernail), and then another non-magnetic section that caps the tip of the artificial fingernail (that extends beyond the end of the finger). In these embodiments, magnetic anchor points are positioned within the nail in order to improve the bond between the artificial nail and the finger, but minimizing the attractive force to external objects (e.g., car and refrigerator doors).

In step 204, a user applies the magnetic nail polish to their existing fingernail. In some embodiments this nail polish is simply allowed to dry as is. In such a configuration, the magnetic particles of the nail polish will likely align themselves to one another resulting in an antiferromagnetic composition or a paramagnetic composition. Where the magnetic particles chosen for the nail polish are “soft” or have a low coercive force, the resulting composition is of little concern as the moment of the dried nail polish can be aligned as desired after drying when in the presence of a hard magnet (such as the artificial nail).

Where the nail polish uses magnetic particles that are hard magnets, the polish may be dried in the presence of a hard magnet (such as the artificial nail) in order to ensure alignment of the particles with the hard magnet. As typical consumer nail polish tends to require 15-60 minutes to dry, it is preferential in some embodiments to use quick drying nail polish preparations. It is contemplated that holding a hard magnet in position for 15 or more minutes may be uncomfortable for some users.

In step 206, the magnetic nail polish is allowed to dry. As noted above, in some embodiments, the drying process is performed in the presence of a hard magnet. In step 208, the user places the artificial nail on the dry nail polish and the magnetic field between the two materials (dried nail polish and artificial nail) affixes on to the other.

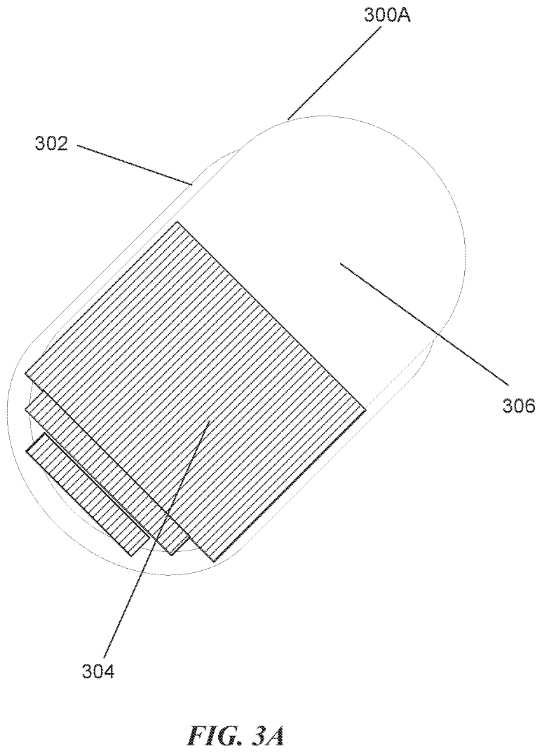

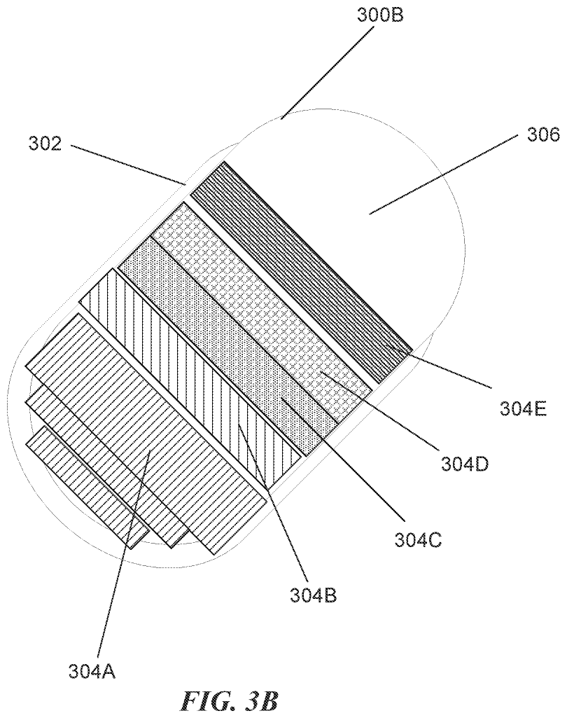

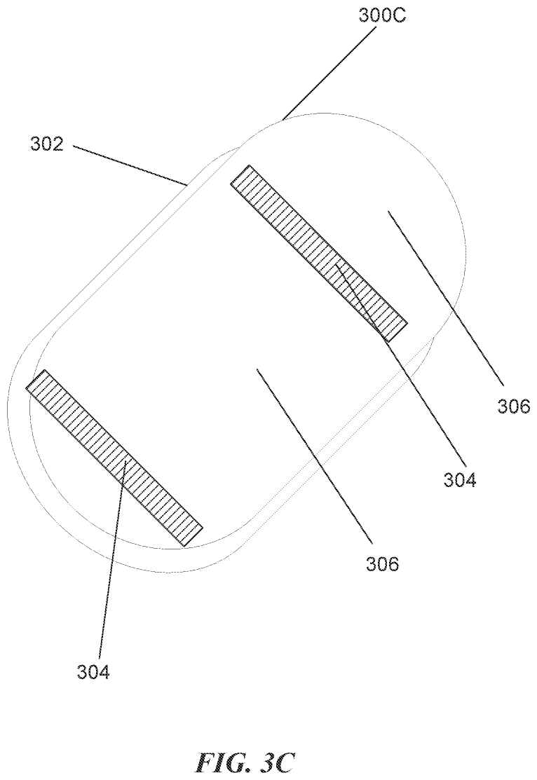

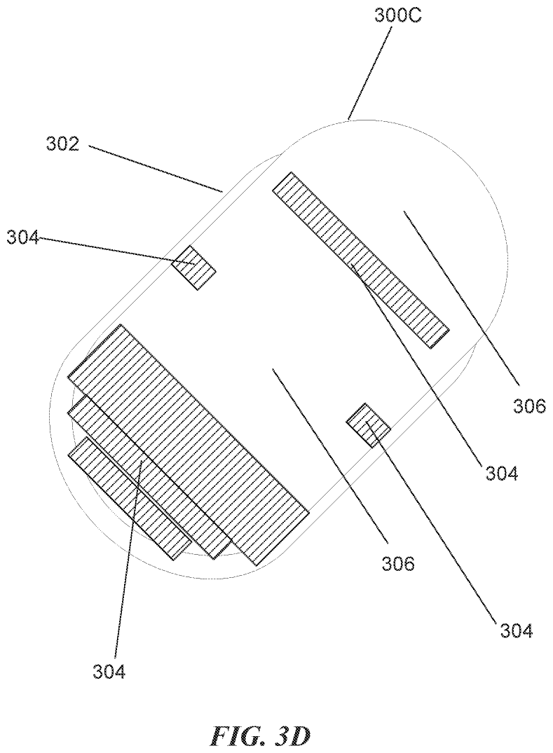

FIGS. 3A-D are depictions of a number of varied configurations of magnetic polymer artificial nails.

FIG. 3A depicts a sharp change in magnetic moment of the artificial magnetic nail. The magnetic artificial nail 300A affixes to a human finger 302 and includes a magnetic portion 304 and a non-magnetic portion 306 (e.g., standard acrylic). The magnetic portion 304 is positioned to be in the lower half of the artificial nail, while the non-magnetic portion 306 is positioned towards the tip. The magnetic moment of the nail has sharp change where the materials are changed.

FIG. 3B depicts a gradual change in magnetic moment of the artificial magnetic nail. The magnetic artificial nail 300B affixes to a human finger 302 and includes multiple varied magnetic portions 304A-E and a non-magnetic portion 306 (e.g., standard acrylic). The magnetic portions 304A-E vary in strength of magnetic moment and gradually reduce from the base to the tip (which is non-magnetic). The manner of reduction of magnetic moment may be by any of concentration of magnetic particles and/or selection of magnetic material. The magnetism exhibited by the artificial nail is thus described by a gradient.

FIG. 3C depicts a couple anchor points of magnetic field of the artificial magnetic nail. The magnetic artificial nail 300C affixes to a human finger 302 and includes two depicted magnetic portions 304 and two non-magnetic portions 306 (e.g., standard acrylic). The magnetic portion 304 is positioned as anchor points at the base of the nail and at the tip of the human finger 302. In this configuration 300C, the smallest amount of magnetic polymer material is used in order to reduce the attractive force of the artificial nail and the external objects (e.g., refrigerators). However, the magnetic polymer material is positioned strategically such that the nail has mechanically advantaged positions on the artificial nail to remain affixed during daily life.

FIG. 3D depicts a number of anchor points of magnetic field distributed throughout the artificial magnetic nail. The magnetic artificial nail 300D affixes to a human finger 302 and includes multiple depicted magnetic portions 304 and two non-magnetic portions 306 (e.g., standard acrylic). Configuration 300D is comparable to configuration 300C; however, configuration 300D includes more magnetic polymer and more anchor points. Configuration 300D illustrates the idea that the artificial nail is highly customizable during manufacture with respect to positioning of the magnetic polymer, and that the position and strength of magnetic material is customizable to the needs of the user.

As will be understood by those familiar with the art, the invention may be embodied in other specific forms without departing from the spirit or essential characteristics thereof. Likewise, the particular naming and division of the members, features, attributes, and other aspects are not mandatory or significant, and the mechanisms that implement the invention or its features may have different names, divisions and/or formats. Accordingly, the disclosure of the invention is intended to be illustrative, but not limiting, of the scope of the invention, which is set forth in the following Claims.

1. A system comprising:
a magnetic nail polish preparation; and
an artificial fingernail including magnetic polymers configured to magnetically attract to a surface including the magnetic nail polish preparation in a dry state.

2. The system of claim 1, wherein the magnetic polymer includes particles of neodymium iron boron.

3. The system of claim 1, wherein a magnetic field associated with the artificial fingernail is stronger on a base end than on a tip end.

4. The system of claim 3, wherein the magnetic polymer consists of multiple magnetic polymers each varying in magnetic moment based on a magnetic moment of particles included in the each of the multiple magnetic polymers.

5. The system of claim 3, wherein the strength of the magnetic field decreases gradually across the length of the artificial fingernail via a reduced concentration of magnetic particles at the tip.

6. The system of claim 3, wherein the strength of the magnetic field decreases sharply at a point along the length of the artificial fingernail via a reduced concentration of magnetic particles at the tip.

7. The system of claim 1, wherein a polarity of the artificial fingernail is fixed during manufacturing thereof.

8. The system of claim 1, wherein a polarity of the magnetic nail polish preparation is set after application and before drying via drying in the presence of a permanent magnet.

9. The system of claim 1, wherein the magnetic nail polish preparation is a soft magnet, and the artificial fingernail is a hard magnet that has sufficient coercive force to set the polarity of the magnetic nail polish preparation after the magnetic nail polish preparation has dried.

10. The system of claim 9, wherein the soft magnet is a nickel/iron alloy.

11. The system of claim 10, wherein the nickel/iron alloy is permalloy.

12. A system comprising:
a magnetic nail polish preparation having a low coercive force and a high moment; and
an artificial fingernail including magnetic polymers configured to magnetically attract to a surface including the magnetic nail polish preparation in a dry state, the magnetic polymer having a high coercive force and a high moment.

13. The system of claim 12, wherein the magnetic polymer includes particles of neodymium iron boron.

14. The system of claim 12, wherein a magnetic field associated with the artificial fingernail has a stronger magnetic field on a base end than on a tip end.

15. The system of claim 14, wherein the magnetic polymer consists of multiple magnetic polymers each varying in magnetic moment based on a magnetic moment of particles included in the each of the multiple magnetic polymers.

16. The system of claim 14, wherein the strength of the magnetic field decreases gradually across the length of the artificial fingernail via a reduced concentration of magnetic particles at the tip.

17. The system of claim 14, wherein the strength of the magnetic field decreases sharply at a point along the length of the artificial fingernail via a reduced concentration of magnetic particles at the tip.

18. A method comprising:
applying a magnetic nail polish preparation to a fingernail;
allowing the applied magnetic nail polish preparation to dry;
affixing an artificial fingernail including magnetic polymers via magnetism.

19. The method of claim 18, further comprising:
manufacturing the artificial fingernail including magnetic polymers with a reduced concentration of magnetic particles at the tip of the artificial fingernail.

20. The method of claim 18, wherein said allowing is performed in the presence of a permanent magnetic that sets the polarity of the magnetic nail polish preparation as the magnetic nail polish preparation dries.
